(19) United States
(12) Patent Application Publication
(10) Pub. No.: US 2026/0000294 A1
(43) Pub. Date: Jan. 1, 2026

(54) AUTOFOCUS CONTROL FOR OPHTHALMIC MICROSCOPE USING LOW-COHERENCE INTERFEROMETRY

(71) Applicant: Alcon Inc., Fribourg (CH)

(72) Inventor: Steven T. Charles, Memphis, TN (US)

(73) Assignee: Alcon Inc., Fribourg (CH)

(21) Appl. No.: 19/248,696

(22) Filed: Jun. 25, 2025

Related U.S. Application Data
(60) Provisional application No. 63/665,565, filed on Jun. 28, 2024.

(51) Int. Cl. A61B 3/13; A61B 3/00; A61B 3/10; A61F 9/007

(52) U.S. Cl. CPC A61B 3/13; A61B 3/0008; A61B 3/102; A61F 9/00736

(57) ABSTRACT

A system for automatically focusing (“autofocusing”) a microscope during an inner limiting membrane (ILM) peeling maneuver is configured to output low-coherence infrared (IR) light toward the eye during the maneuver. The system includes an interferometer that senses an axial distance between the microscope and ILM and outputs distance signals indicative of the sensed axial distance. An electronic control unit (ECU) is in communication with motor-driven lenses of the microscope and with an IR light source of the interferometer. Execution of the instructions causes the ECU to receive the distance signals from the interferometer. In response to the distance signals, the ECU transmits a focus control signal to the lens(es) to thereby resolve the ILM with a desired depth-of-field. The ECU automatically adjusts a focus level of the microscope on the ILM to accommodate for respiration-related movements of the patient.

### CROSS-REFERENCE TO RELATED APPLICATIONS

The present application claims the benefit of priority to U.S. Provisional Application No. 63/665,565 filed on Jun. 28, 2024, which is hereby incorporated by reference in its entirety.

### INTRODUCTION

The intricate anatomy of the human eye is adapted to focus light on the retina so that the brain is able to perceive images and colors. Ambient light enters the anterior chamber and vitreous chamber through the cornea and pupil. The cornea and a natural or artificial lens situated behind the iris together focus the light onto sensitive photoreceptor cells of the retina, which in turn is located on the posterior wall of the vitreous chamber. Light transits the vitreous chamber through a transparent gelatinous fluid referred to as the vitreous humor. The light is converted into electrical signals upon reaching the retina. The electrical signals from the retina are then communicated to the brain stem via the optic nerve. The brain thereafter converts the received electrical signals into the perceptible images and colors noted above, i.e., sight.

The inner surface/vitreous side of the retina is covered by a thin transparent layer of tissue referred to as the internal limiting membrane (ILM). An intact and healthy ILM performs a host of beneficial functions, including serving as a basement membrane for the eye's Müller cells. During an ILM peeling maneuver, a surgeon carefully lifts the ILM and peels it away from the retina to eliminate traction on the macula. Optimal ILM visualization, even during vertical respiration-driven motion, is required during a host of vitreomacular surgeries, including but not limited to microsurgical repairs of macular holes, macular schisis, and epimacular membranes.

### SUMMARY

Disclosed herein is an automatically focusing (“autofocusing”) system and associated method for controlling an ophthalmic microscope during a vitreomacular surgery, specifically one requiring the performance of an internal limiting membrane (ILM) peeling procedure or “ILM peel”. As set forth herein, the axial distance between the ILM and a focusing lenses of the microscope is performed using low-coherence interferometry for the purpose of controlling a motor-driven focusing function of the microscope. The solutions described below enable the microscope to automatically maintain its focus on the ILM and also center the microscope's depth-of-field while the surgeon views the ILM. Focus on the ILM and the surgeon's desired/predetermined depth-of-field are automatically maintained in spite of motion of the ILM, with such motion caused, for example, by the patient's respiration and/or other involuntary or voluntary movements of the patient.

Aspects of the present disclosure pertain to the use of a low-coherence infrared (IR) light source, for instance at least one super luminescent light-emitting diode (SLED), to illuminate the ILM. As contemplated herein, the IR light source is an integral component of the interferometer. The ILM is the first reflective surface encountered by the low-coherence IR light upon the IR light entering the vitreous chamber after first passing through the cornea, pupil, and lens. Backscattered IR light is sensed via an interferometer and output as a voltage or another application-suitable electrical or electronic distance signals to an electronic control unit (ECU). The ECU thereafter uses the sensed axial distance, represented by the received distance signals, to adjust focus levels of one or more motor-driven lenses of the microscope. The microscope therefore automatically accommodates or adapts to respiration-related and other movements of the patient during the vitreomacular surgery.

The autofocusing system according to a representative implementation includes a digital or analog ophthalmic microscope having one or more of the aforementioned motor-driven lenses. The interferometer may be embodied as an axial-scan ocular coherence tomography (A-scan OCT) device or a swept-source biometer in different exemplary implementations.

As part of this representative construction, the ECU is in wired and/or wireless communication with the microscope and the interferometer. The ECU includes a processor and a non-transitory, computer-readable storage medium (“memory”). The memory of the ECU is programmed with computer-readable instructions, the execution of which by the processor in response to an input signal causes the ECU to receive the distance signals that are outputted by/from the interferometer. In response to receipt of the distance signals, the ECU transmits a focus control signal (or signals) to the motor-driven lens(es) wirelessly or over physical transfer conductors, e.g., voltage signals, to cause the microscope to resolve the ILM with a desired depth-of-field. Autofocus occurs with minimal latency, for instance with less than about 5-10 milliseconds (ms) of latency in one or more implementations, or at least significantly shorter than the typical 400 ms of latency experienced during manual focusing. Such a response is considered herein, for surgical purposes, to be nearly instantaneous in comparison to manual focus alternatives.

Aspects of the present disclose are also directed to a user interface that is in communication with the noted ECU and configured to generate the input signal. Possible embodiments of the user interface include a surgeon-activated/manually-actuated foot pedal, a touch-sensitive surface, and/or voice recognition software configured to generate the input signal in response to voice commands.

Continuing with the present summary, a method for automatically focusing on the ILM during a vitreomacular surgery includes sensing the axial distance between the microscope and the ILM in response to an input signal from the aforementioned user interface. Sensing of the axial distance is performed using the interferometer as the ILM is illuminated with low-coherence IR light from the low-coherence IR light source. This action may occur while the surgeon illuminates the vitreous chamber with white light using an endoilluminator tool or chandelier. The method may include outputting one or more distance signals from the interferometer.

One or more embodiments include receiving the distance signal(s) from the interferometer via the ECU. In response to the distance signals, the method includes transmitting focus control signals to a motor-driven lens, lenses, or lens cells of the microscope. The microscope is thereby caused to resolve the ILM with a desired/predetermined depth-of-field and automatically adjust a focus level of the microscope on the ILM to accommodate for respiration-related movements of the patient during the vitreomacular surgery. The disclosed autofocus function is automatic in the sense that the ILM remains in focus without surgeon intervention.

A representative implementation of the ECU includes the processor and memory, the latter of which is programmed with computer-readable instructions. The programmed instructions are executable by the processor to cause the ECU to command a low-coherence IR light source to output and direct low-coherence near-infrared (NIR) light toward an eye during a vitreomacular surgery, and to command an A-scan OCT device or swept-source biometer to sense the axial distance between the microscope and an ILM of the eye. Execution of the instructions also causes the ECU to receive a distance signal from the A-scan OCT device or swept-source biometer indicative of the axial distance, and to transmit focus control signals to at least one motor-driven lens of the microscope.

The above-described features and advantages and other possible features and advantages of the present disclosure will be apparent from the following detailed description of the best modes for carrying out the disclosure when taken in connection with the accompanying drawings.

### BRIEF DESCRIPTION OF THE DRAWINGS

FIG. 1 is an illustration of a representative surgical suite having an automatic focusing (“autofocusing”) system configured for use during a vitreomacular surgery requiring performance of an internal limiting membrane (ILM) peeling maneuver.

FIGS. 2A and 2B illustrate a representative ILM peeling maneuver that may be performed with the assistance of the autofocusing system shown in FIG. 1.

FIG. 3 is a representative configuration of the autofocusing system in accordance with an exemplary embodiment.

FIG. 3A illustrates an embodiment of the autofocusing system of FIG. 3 in further detail.

FIG. 4 is a flow chart describing a method for focusing on the ILM of FIGS. 2A and 2B using the autofocusing system illustrated in FIG. 3.

The solutions of the present disclosure may be modified or presented in alternative forms. Representative embodiments are shown by way of example in the drawings and described in detail below. However, inventive aspects of this disclosure are not limited to the disclosed embodiments. Rather, the present disclosure is intended to cover alternatives falling within the scope of the disclosure as defined by the appended claims.

### DETAILED DESCRIPTION

Embodiments of the present disclosure are described herein. The disclosed embodiments are examples that may take alternative forms. The Figures are not necessarily drawn to scale. Some features may be exaggerated or minimized to show details of particular components. Therefore, specific structural and functional details disclosed herein are non-limiting and act as a representative basis for teaching one skilled in the art to variously employ the present disclosure. As those of ordinary skill in the art will appreciate, the various features illustrated and described with reference to any one of the Figures can be combined with features illustrated in one or more other Figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Certain terminology may be used in the following description for the purpose of reference only, and thus are not intended to be limiting. For example, terms such as “above” and “below” refer to directions in the drawings to which reference is made. Terms such as “front,” “back,” “fore,” “aft,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

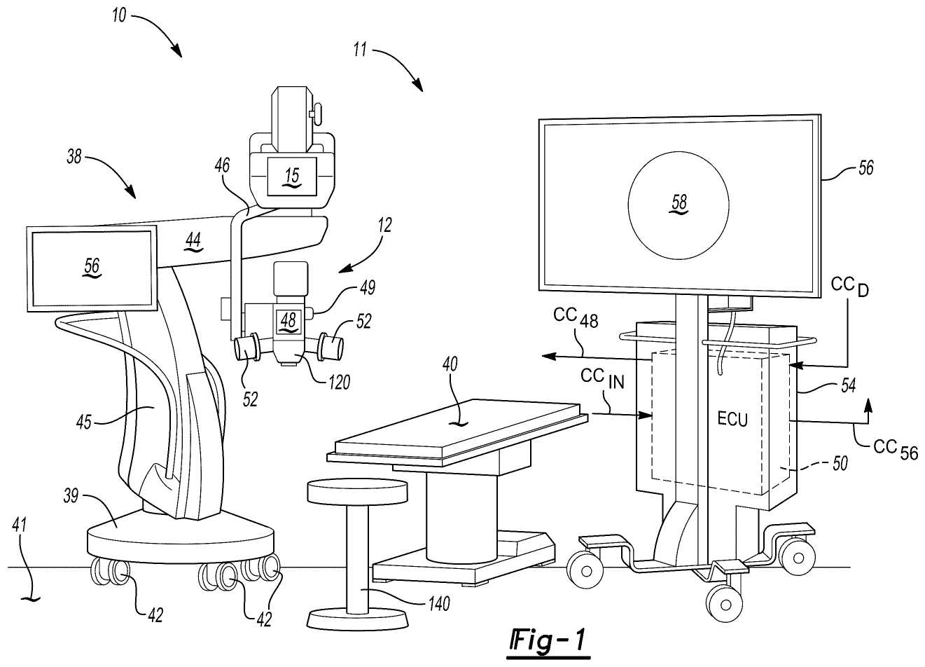

Referring to the drawings, wherein like reference numbers refer to like components, a representative surgical suite 10 is illustrated in FIG. 1. As described below, the surgical suite 10 is equipped with an automatically focusing (“autofocusing”) system 11 equipped with an interferometer 15 and an electronic control unit (ECU) 50. The interferometer 15 and ECU 50 together enable a surgeon (not shown) to enjoy autofocusing functionality of an ophthalmic microscope 12 while performing an internal limiting membrane (ILM) peeling maneuver during a vitreomacular surgery.

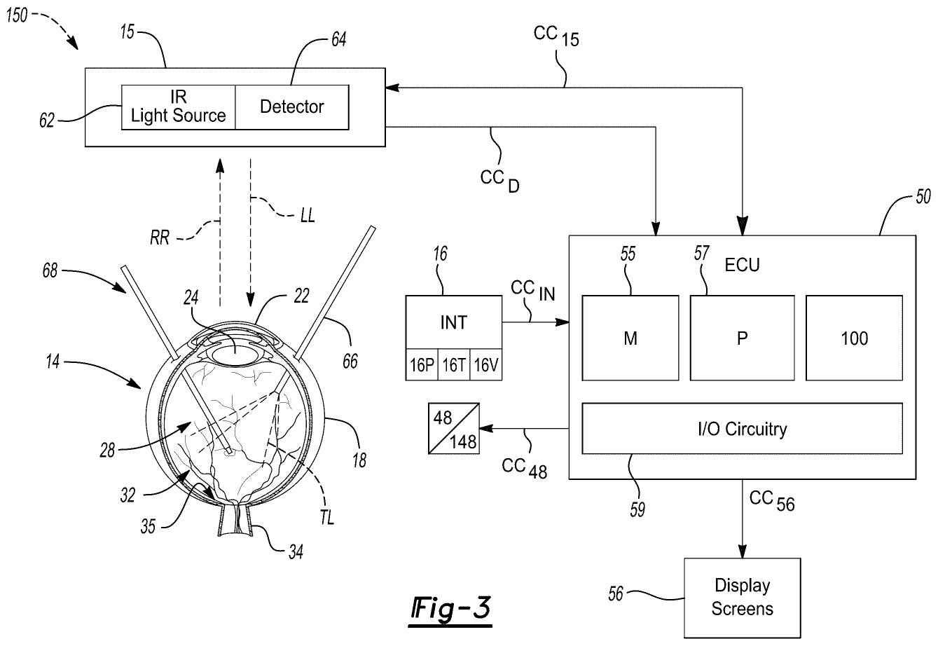

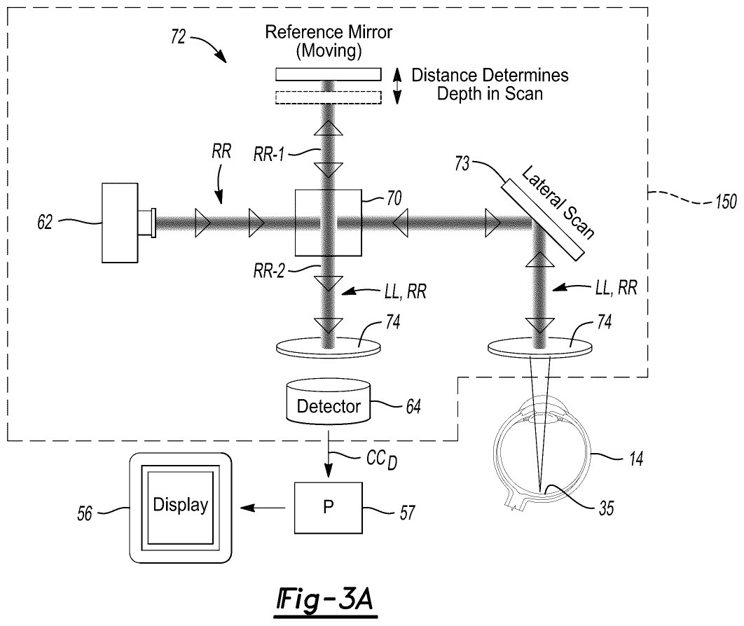

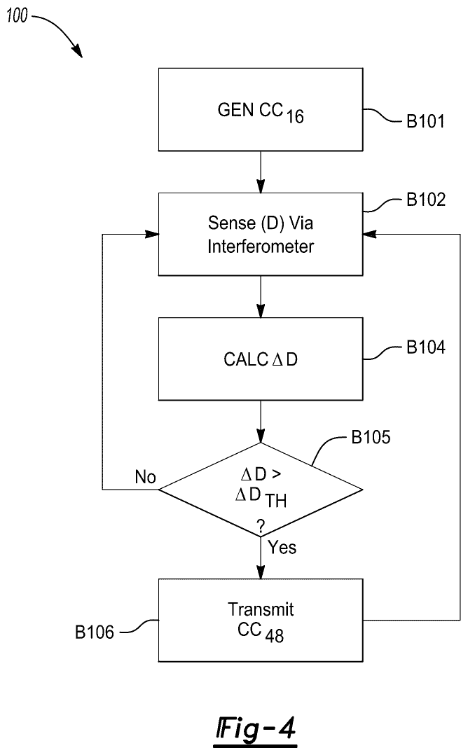

Autofocusing functionality of the microscope 12 is enabled by use of the interferometer 15 and its emitted low-coherence infrared (IR) light as set forth in detail below with reference to FIGS. 3, 3A, and 4.

The ILM's delicate structural composition renders the ILM difficult to resolve using standard ocular coherence tomography (OCT) or other medical visualization methodologies. A typical OCT system has a resolution of about 5-20 micrometers (μm). In comparison, the ILM has a thickness of less than about 3 μm at its thickest point near the central fovea. Even if OCT resolution were to somehow increase, the necessary adjustments would decrease the depth-of-field. As appreciated in the art, depth-of-field refers to the distance between the nearest and farthest object planes when both planes are in focus. In microscopy, depth-of-field thus defines how far above and below a sample plane an objective lens and the specimen can be while remaining in perfect focus.

Undesirable reduction in depth-of-field during an ILM peel may render real-time detection of the ILM impracticable. This is due largely to the multitude of voluntary or involuntary patient movements that can occur during a typical vitreomacular surgery. Particularly problematic is the rhythmic vertical body motion imparted by a patient's normal respiration as well as more abrupt movements due to, e.g., coughing, sneezing, or dozing/waking during the surgery. The present teachings therefore pertain to automatic adjustment of a focus level of the microscope 12 on the ILM to accommodate or adapt to a patient's respiration-related movements during a vitreomacular surgery.

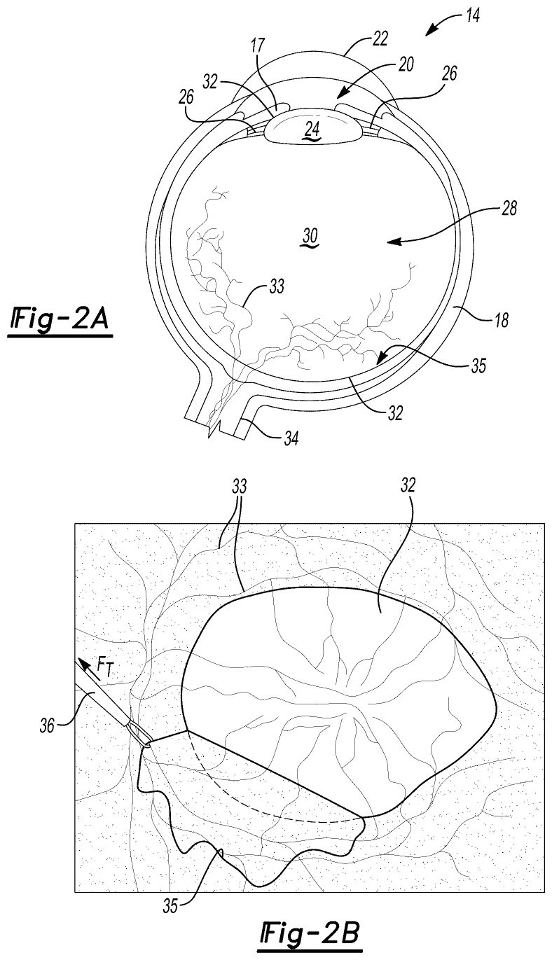

Referring briefly to FIG. 2A, a human eye 14 includes an iris 17 surrounded by sclera 18. A pupil 20 is centrally located within/surrounded by the iris 17. A dome-like cornea 22 spans and protects the iris 17 and pupil 20. Light admitted through the pupil 20 passes through a lens 24, with the lens 24 in turn connected to the surrounding anatomy via ciliary muscles 26. Also shown in FIG. 2A is the vitreous chamber 28. This roughly spherical volume of the eye 14 is filled with a gelatinous fluid referred to as the vitreous humor 30. A retina 32 lines a posterior wall of the vitreous chamber 28 and is largely surrounded by a network of blood vessels 33, with an ILM 35 lining the vitreous side of the retina 32. An optic nerve 34 is disposed in the posterior region of the eye 14 approximately opposite the lens 24 to carry electrical signals to the brain stem (not shown) as summarized above.

In FIG. 2B, a portion of the eye 14 is illustrated while the eye 14 undergoes an ILM procedure during which the ILM 35 is carefully separated and peeled away from the retina 32 as summarized above. During an ILM peel, a surgeon wields a surgical tool 36 to impart gentle traction forces (arrow Fr) to the ILM 35 after performing initial incisions. In this manner, the surgeon is able to peel back the ILM 35 to expose as much of the retina 32 or macular regions as is needed when preparing for a particular vitreomacular surgery. For example, an ILM peel may involve the use of a forceps-assisted “pinch-and-peel” technique or a friction-based technique, with the latter utilizing a specialized scraping loop such as the FINESSE™ Flex Loop Curved Nitinol loop commercially from Alcon, Inc. Thus, the identity of the surgical tool 36 varies with the particular portion of the ILM peel being performed.

The ILM 35 has a maximum thickness of about 3 μm as noted above and thins to about 1 μm nearest the central macula or fovea. In other words, the ILM 35 is exceptionally thin. The ILM 35 is also transparent. The thinness and transparency of the ILM 35 often results in its movement due to imparted forces, for instance involuntary movements due to the patient's respiration or voluntary and/or transferred head or eye movements. ILM peeling from the standpoint of required surgical dexterity and visualization is thus considered to be one of the more difficult surgical tasks in modern vitreoretinal surgery.

Visibility of the ILM 35 is often facilitated by use of a small amount of contrast-enhancing staining dye such as indocyanine green (ICG) or Membrane-Blue-Dual (DORC International), which lightly stains the ILM 35 to improve contrast. While the dye helps the surgeon maintain visibility of the ILM 35, the ILM 35 is often in motion during the surgery. The surgeon is therefore required, absent the present teachings, to manually focus the microscope 12 of FIG. 1 to maintain a desired focus level and depth-of-field on the dynamic ILM 35. However, the need to manually adjust the microscope 12 to keep the ILM 25 in proper focus extends the duration of the surgical task and may also lead to other potential complications. The autofocus-enabling features of the present disclosure avoid the need to manually focus the microscope 12 and thereby help avoid problems commonly associated with such an approach.

As appreciated in the art, autofocus of a single lens reflective (SLR) camera is driven by sensing high spatial frequency detail in a given image. This works quite well when the subject has the requisite detail. Were such a method to be used to image the ILM 35 with its above-described thinness and transparency, however, the microscope 12 of FIG. 1 without the benefit of the present teachings would tend to detect and focus on the blood vessels 33 of FIGS. 2A and 2B, the retinal pigment epithelium, i.e., the pigmented cells of the outermost layer of the retina 32 of FIGS. 2A and 2B, or other ocular features having a relatively high spatial frequency detail, as opposed to focusing on the barely perceptible ILM 35. The present disclosure thus enables autofocus of the microscope 12, specifically by using low-coherence interferometry in the manner described in detail below.

Referring again to FIG. 1, the representative surgical suite 10 may be equipped with a surgical robot 38 and an operating platform 40, for example a table as shown or a reclinable chair. During performance of a vitreomacular surgical procedure, a patient (not shown) may be positioned on the operating platform 40. A surgeon (also omitted for illustrative simplicity) may stand near the operating platform 40 or remain seated on a stool 140 or another suitable seating surface. The robot 38 may be mounted or positioned relative to a floor 41 of the surgical suite 10, for instance directly or via a mobile platform 39 having lockable wheels 42. The mobile platform 39 in the illustrated exemplary embodiment is connected to via an intervening support column 45 to an articulated robot arm 44, for instance a six degree-of-freedom surgical robot arm. The microscope 12 in such a configuration is mounted to the articulated robot arm 44, for instance via a support bracket 46.

The representative ophthalmic microscope 12 of FIG. 1 consists of several main components. An optical head 120 of the microscope 12 contains one or more motor-driven lenses 48 or lens cells 148 (FIG. 3) and associated optics for magnifying the eye 14 of FIG. 2A during a vitreomacular surgery. Lens 48 is used hereinafter in the singular solely for illustrative consistency, with “a” meaning “one or more” unless otherwise specified. Shown schematically for illustrative simplicity and clarity, the optical head 120 may contain an objective lens and zoom section providing various levels of magnifications, a pair of eyepieces 49 (a portion of one of which is visible in FIG. 1) through which the surgeon views magnified images of the eye 14, and a microscope light source (not shown). The microscope 12 may also be equipped with handles 52 to facilitate maneuvering or positioning of the microscope 12 by the surgeon, with gross positioning provided by the robot 38 in one or more embodiments, e.g., via multi-axis motorized joint control as appreciated in the art.

Also present within the exemplary ophthalmic surgical suite 10 of FIG. 1 is a cabinet 54 containing the above-noted ECU 50. The optional cabinet 54 may be constructed of a lightweight and easily sanitized construction, e.g., painted aluminum or stainless steel, and used to protect constituent hardware of the ECU 50 from possible ingress of dust, debris, and moisture. The ECU 50 may be embodied as one or more computer devices configured as described below with reference to FIG. 3 to coordinate electronic features and settings of the microscope 12 and other equipment or payloads used within the surgical suite 10. Autofocus control of the microscope 12 of FIG. 1 via the ECU 50 occurs by wired or wireless transmission of focus control signals (CC₄₈) to one or more of the lenses 48 of the microscope 12 as noted above.

Still referring to FIG. 1, the surgical suite 10 may also include high-resolution display screens 56 to enable a surgeon and attending medical team/technicians to view projected images 58 of the surgical scene, in this instance the retina 32 and the ILM 35 of FIG. 2B. To that end, the ECU 50 is configured to output display control signals (CC₅₆) to the display screens 56 to enable images of the surgical scene to be clearly displayed. In different implementations, the surgeon/surgical team may view such projected images 58 as three-dimensional images using a head up display system, polarized glasses, virtual reality glasses, or the eyepieces 49. In particular, the ECU 50 is configured to receive electronic or electrical distance signals (CC_{D}) from the interferometer 15 in response to an input signal (CC_{IN}) as set forth below, with the distance signals (CC_{D}) allowing the microscope 12 to maintain focus on the ILM 35 of FIG. 2B.

Referring now to FIG. 3, the autofocusing system 11 is shown in operation during a vitreomacular surgery of the eye 14. When a surgeon requires the microscope 12 to autofocus on the ILM 35 of FIG. 2B, the surgeon may request or trigger generation of the input signal (CC_{IN}). A user interface (INT) 16 may be included within the structure of the autofocusing system 11 in one or more embodiments, with the user interface 16 being in wired and/or wireless communication with the ECU 50.

In various embodiments, the user interface 16 may include a manually-actuated foot pedal 16P, a touch-sensitive surface 16T, for instance a capacitive touchscreen or surface of one of the display screens 56 of FIG. 1 or another screen, and/or voice recognition software 16V optionally configured to generate the input signal (CC_{IN}) in response to voice commands. In response to the surgeon-requested or auto-generated input signal (CC_{IN}), the autofocusing system 11 of FIG. 3 initiates low-coherence sensing of an axial distance between the microscope 12 of FIG. 1 and the ILM 35 of the eye 14. The ILM 35 is the first optical reflective surface encountered by light from the interferometer 15 as the infrared light noted below traverses the vitreous cavity 28 beyond the lens 24.

As contemplated herein, the interferometer 15, the microscope 12 (FIG. 1), or another component of the autofocusing system 11 is equipped or used in conjunction with a low-coherence infrared (IR) light source 62 operable for transmitting a low-coherence IR light as a light beam (LL) toward the ILM 35. The IR lighting spectrum is generally divided into near-IR (NIR) light, with wavelengths of about 0.78 μm to about 2.5 μm, middle IR light (about 2.5 μm to about 15 μm), and far IR light (about 15 μm to about 1 mm). In one or more embodiments, the lighting spectrum of the IR light beam (LL) includes near-infrared light provided by the IR light source 62 of FIG. 3, which may occur while an endoillumination tool 66 such as a chandelier is used to direct white light. The lighting spectrum from the low-coherence IR light source 62 may be exclusively NIR light in its composition in some implementations. The surgeon may select a desired lighting spectrum via the ECU 50 in a possible implementation, e.g., using the user interface 16.

LOW-COHERENCE INTERFEROMETRY: As noted above, low-coherence interferometry is used herein to sense the axial distance between the microscope 12 of FIG. 1 and the ILM 35. The term “temporal coherence” refers to characterization of the temporal continuity of a wave train, in this instance IR light, that is sent out by the low-coherence IR light source 62 and measured by the detector 64. Although the level of coherence may vary with the application, “low-coherence” as used herein may refer to a coherence wavelength on the order of, e.g., tens of microns or less. Sufficiently low-coherence for the purposes of the present autofocusing system 11 may be achieved using a super-luminescent light emitting diode (SLED) emitting photons that are not in phase. As a result, low-coherence interferometry enables high depth resolution due to the relatively short coherence length.

The ECU 50 thereafter uses the sensed axial distance represented by the distance signals (CC_{D})) to control an autofocus setting of the microscope 12, for instance by adjusting a state of the motor-driven lens 48 (or lens cells 148) via focusing control signals (CC₄₈) to maintain focus of the ILM 35 and center a desired depth-of-field. Autofocusing control occurs without intervention by the surgeon. Instead, the interferometer 15 and ECU 50 of FIG. 3 monitor changes in interference patterns produced by overlapping out-of-phase waves as the low-coherence IR light beam (LL) reflects off of the ILM 35 as reflected light (RR), with the sensed reflection or interference patterns corresponding to the axial distance.

As appreciated in the art, low-coherence interferometry involves the direction of low-coherence light at a given target, in this case low-coherence IR light directed at the ILM 35, and the detection of reflected light/interference patterns via a suitable detector 64. The detector 64 in one or more embodiments may include a charge-coupled device image sensor or another application-suitable sensor operable for converting patterns of optical interference fringes into electrical distance signals (CC_{D}) indicative of the sensed axial distance.

Referring briefly to FIG. 3A, in a possible implementation suitable for sensing the axial distance, the interferometer 15 of FIG. 3 may be optionally constructed as a time-domain or frequency domain axial-scan ocular coherence tomography (A-scan OCT) device 150. The A-scan OCT device 150 is in communication with the above-described display screen(s) 56 and the processor/CPU 57 of the ECU 50. The A-scan OCT device 150 may include the low-coherence IR light source 62, a beam splitter 70, a moveable reference mirror 72, a lateral scan mirror 73, and lenses 74. The light source 62 and detector 64 for use in the low-coherence interferometry setup of FIG. 3A are in a fixed axial mechanical relationship to each other and the microscope 12 of FIG. 1.

For the purposes of the present application, i.e., A-scan (single-line OCT), the mirror 73 is not moved laterally in a plane. Here, the IR light beam (RR) from the IR light source 62 is directed toward the beam splitter 70. Some of the incident IR light beam (RR) passes through the beam splitter 70 toward the mirror 73, and from the mirror 73 is directed toward the ILM 35 through lens 74. Another lens 74 directs light from the beam splitter 70 onto the detector 64. The distance signal (CC_{D})) is thereafter communicated to the processor 57 as set forth herein. Other implementations of the interferometer 15 may be used in other implementations. For instance, the interferometer 15 of FIG. 3 may be constructed as a swept-source biometer, e.g., the commercially available ARGOS® Swept-Source OCT Biometer from Alcon, Inc.

The ECU 50 in the particular construction of FIG. 3 is in communication with the microscope 12 (FIG. 1) and the interferometer 15. Execution of instructions, which are programmed in/recorded on a non-transitory computer-readable storage medium or memory 55, by a processor 57 occurs in response to the input signal (CC_{IN}) and causes the ECU 50 to receive the distance signals (CC_{D}) from the interferometer 15. In response to the distance signals (CC_{D}), the ECU 50 is caused to transmit the focus control signals (CC₄₈) to the motor-driven lens(es) 48 to cause the microscope 12 to resolve the ILM 35 with a desired depth-of-field, e.g., of the surgeon or a preset or preselected resolution. Such a desired depth-of-field may be programmed into the memory 55, for instance customizable to the preferences of a given surgeon.

Although the ECU 50 is depicted as a unitary box in FIG. 3 for illustrative clarity and simplicity, the ECU 50 within the scope of the disclosure may include one or more networked devices each with a central processing unit or other processor (P) 57 and sufficient amounts of memory (M) 55, including a non-transitory (e.g., tangible) medium that participates in providing data/instructions that may be read/executable by the processor(s) 57. Instructions may be stored in the memory 55 and executed by the processor 57 to perform the various visualization functions described herein, thus enabling the present method 100 as exemplified in FIG. 4.

The memory 55 of FIG. 3 may take many forms, including but not limited to non-volatile media and volatile media. Non-volatile media may include optical and/or magnetic disks or other persistent memory, while volatile media may include dynamic random-access memory (DRAM), static RAM (SRAM), etc., any or all which may constitute a main memory of the ECU 50. Input/output (I/O) circuitry 59 may be used to facilitate connection to and communication with the various peripheral devices used during the ophthalmic procedure, inclusive of the various hardware of the autofocusing system 11 of FIG. 3.

Other hardware not depicted but commonly used in the art may be included as part of the ECU 50, including but not limited to a local oscillator or high-speed clock, signal buffers, filters, etc. The ECU 50 may control the microscope 12 of FIG. 1, e.g., via the focus control signals (arrow CC₄₈). Display control signals (CC₅₆) are directed to the display screens 56 as described above. The ECU 50 may also be configured to communicate with the interferometer 15 via interferometer control signals (CC₁₅) in response to the input signals (CC_{IN}), e.g., for instance using a serial bus, a local area network, a controller area network, a controller area network with flexible data rate, or via Ethernet, Wi-Fi, Bluetooth™, near-field communication, and/or other forms of wired or wireless data connection.

Communication of the various signals contemplated herein may be accomplished using one or more direct-wired links, a networked communications bus link, a wireless link, a serial peripheral interface bus, and/or other suitable communications links. Communication may include exchanging data signals in a suitable form, including but not limited to electrical signals via a conductive medium, electromagnetic signals via air, optical signals via optical waveguides, and the like. Data signals may include signals representing inputs from sensors, signals representing actuator commands, and communications signals between controllers.

Still referring to FIG. 3, during the vitreomacular surgery, task lighting and location of the ILM 35 may be facilitated by an endoilluminator 66. The endoilluminator may be coupled to an accompanying filtered power supply (not shown) such as a filtered wall outlet or a battery pack and power inverter suitable for ensuring reliable generation and transmission of task lighting (TL). The surgeon may also insert the surgical tool 68 into the vitreous cavity 28 to perform the above-summarized ILM peel. As this occurs, the IR light beam (LL) from the IR light source 62 passes through the cornea 22 and lens 24 when sensing the axial distance between the microscope 12 of FIG. 1 and ILM 35 as noted above. The task lighting (TL) emitted by the endoilluminator 66 is white/visible light that aids the surgeon when viewing the ILM 35. Control of the lenses 48 follows as needed based on the axial distance.

The lenses 48 contemplated herein may be optionally constructed as moveable motorized lenses or lens cells 148, e.g., achromatic lenses or apochromatic lenses or lens groups. Such lenses 48 or 148 allow the surgeon to adjust focal length or magnification levels in real time by moving, via linear or rotary actuators of the microscope 12, closer or farther away from each other and/or the ILM 35. The motorized lenses or lens cells 148 as contemplated herein reduce focusing latency to several milliseconds. In one or more embodiments, for instance, the motor-driven lenses 48 or lens cells 148 may configured to focus to about +/−10 diopters in less than about 2 ms, less than about 5-10 ms, or at least with significantly reduced latency relative to the 400 ms latency characteristic of manual focusing alternatives.

FIG. 4 illustrates a possible embodiment of the method 100 for autofocusing the microscope 12 of FIG. 1 on the ILM 35 of FIG. 2B. FIG. 4 is described in terms of computer-readable instruction/code segments or logic blocks for clarity, with each constituent logic block of the method 100 being executable by the processor 57 of the ECU 50 during a given vitreomacular surgery.

Beginning with block B101 (“GEN CCI”), the method 100 includes generating the input signal (CC_{IN}) using the user interface 16 of FIG. 3, e.g., one or more of the foot pedal 16P, the touch-sensitive surface 16S, and/or voice commands to the voice recognition software 16V. The method 100 then proceeds to block B102.

Block B102 (“Sense (D) Via Interferometer”), which is performed in response to the input signal (CC_{IN}) of block B101, includes sensing the axial distance between the ophthalmic microscope 12 and the ILM 35 using the interferometer 15, for instance the A-scan OCT device 150 (or a swept-source biometer). This distance is represented as (D) in FIG. 4. Sensing occurs while simultaneously directing the low-coherence IR light beam (LL) from the low-coherence IR light source 62 toward the eye 14. Directing the low-coherence IR light beam (LL) in block B102 in one or more embodiments may include directing low-coherence near-infrared (NIR) light toward the eye 14, for example using a super luminescent diode (SLED) of the IR light source 62 as noted above. The method 100 thereafter proceeds to block B104.

At block B104 (“CALC AD”), the method 100 of FIG. 4 includes outputting the distance signals (CC_{D}) via the interferometer 15, with the distance signals (CC_{D})) being indicative or representative of the sensed axial distance between the microscope 12 and the ILM 35. Block 104 includes receiving the distance signals (CC_{D}) from the interferometer 15 via the ECU 50, for instance as a voltage signal that the ECU 50 may interpreted as or translated into a corresponding axial distance (D), e.g., using a lookup table. The method 100 then proceeds to block B105.

Block B105 (“ΔD>ΔD_{TH}?”) entails comparing the sensed axial distance from block B104 to a prior sensed axial distance, i.e., a last sensed axial distance, to determine whether the present focus level of the microscope 12 of FIG. 1 is sufficient. The method 100 proceeds to block B106 when a change in the axial distance relative to the prior distance, i.e., ΔD, exceeds the threshold distance variation (ΔD_{TH}). The method 100 proceeds in the alternative to block B102 when the change in the sensed axial distance (ΔD) since the last time step is within threshold distance variation (ΔD_{TH}).

At block B106 (“Transmit CC₄₈”), the ECU 50 responds to the distance signals (CC_{D})) by transmitting the focus control signals (CC₄₈) to the lenses 48 (or 148) of the microscope 12 to cause the microscope 12 to autofocus on the ILM 35 and thereby resolve the ILM 35 with a surgeon's desired depth-of-field. This action may occur for analog as well as digital constructions of the microscope 12, i.e., the microscope 12 may be an analog microscope 12 or a digital microscope 12. As part of block B106, the microscope 12 may resolve the ILM 35 to +/−10 diopters or another application suitable magnification level, e.g., in less than about 2-10 ms, with virtually no image impairment, and without intervention by the surgeon.

Using the autofocusing system 11 of FIG. 1 and its interferometer 15 in the manner described above, a surgeon or technician can selectively activate low-coherence, IR light-based sensing of the axial distance between the ILM 35 of FIGS. 2A, 2B, and 3 and the microscope 12 of FIG. 1. When the method 100 is activated in response to the input signal (CC_{IN}), the ECU 50 would coordinate focusing of the lenses 48 on the ILM 35 with reduced latency. Use of a low-coherence NIR light beam as part of the present strategy will ensure that the surgeon does not see the sensing beam. Optics and anti-reflective coatings may be integrated into the autofocusing system 11 to allow both visible and low-coherence IR wavelengths as needed. Among other potential benefits, the present teachings are intended to improve surgical results and prevent complications sometimes associated with ILM peeling, including retinal dimples or other retinal defects, disassociated nerve fiber layer (DONFL), Müller cell damage, and bleeding. These and other attendant benefits will be readily appreciated by those skilled in the art having the benefit of the present disclosure.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

What is claimed is:

1. An automatically focusing (“autofocusing”) system for use in performing an internal limiting membrane (ILM) peeling maneuver on an eye of a patient, the autofocusing system comprising:
an ophthalmic microscope having a motor-driven lens;
an interferometer configured having a low-coherence infrared (IR) light source and a detector, the detector being operable to:
sense an axial distance between the microscope and an ILM of the eye as a sensed axial distance when the eye is illuminated with low-coherence IR light from the low-coherence IR light source; and
output distance signals indicative of the sensed axial distance; and
an electronic control unit (ECU) in communication with the microscope and the interferometer, the ECU having a processor and a non-transitory, computer-readable storage medium (“memory”) programmed with instructions, wherein execution of the instructions by the processor from the memory in response to an input signal causes the ECU to:
receive the distance signals from the interferometer;
generate focus control signals based on the distance signals; and
transmit the focus control signals to the motor-driven lens in response to the distance signals to thereby cause the microscope to:
resolve the ILM with a predetermined depth-of-field; and
automatically adjust a focus level of the microscope on the ILM to accommodate for respiration-related movements of the patient during the ILM peeling maneuver.

2. The autofocusing system of claim 1, wherein a lighting spectrum of the low-coherence IR light includes near-infrared light.

3. The autofocusing system of claim 1, wherein the low-coherence IR light source includes one or more super luminescent light-emitting diode (SLEDs).

4. The autofocusing system of claim 1, wherein the interferometer is configured as an axial-scan ocular coherence tomography (A-scan OCT) device.

5. The autofocusing system of claim 1, wherein the interferometer is configured as a swept-source biometer.

6. The autofocusing system of claim 1, wherein the motor-driven lenses are configured to resolve the ILM with latency of less than about 10 milliseconds.

7. The autofocusing system of claim 1, further comprising:
a user interface in communication with the ECU, wherein the user interface is configured to generate the input signal.

8. The autofocusing of claim 7, wherein the user interface includes a manually-actuated foot pedal.

9. The autofocusing system of claim 7, wherein user interface includes voice recognition software configured to generate the input signal in response to voice commands.

10. The autofocusing system of claim 1, wherein the ophthalmic microscope is a digital microscope.

11. A method for automatically focusing (“autofocusing”) on an inner limiting membrane (ILM) of an eye of a patient during an ILM peeling maneuver, comprising:
in response to an input signal, sensing an axial distance between an ophthalmic microscope and an ILM of the eye using an interferometer while low-coherence infrared (IR) light is directed toward the eye via a low-coherence IR light source of the interferometer, the ophthalmic microscope having a motor-driven lens;
outputting one or more distance signals, via the interferometer, indicative of the axial distance between the ophthalmic microscope and the ILM;
receiving the one or more distance signals from the interferometer via an electronic control unit (ECU); and
in response to the one or more distance signals, transmitting a focus control signal, via the ECU, to the motor-driven lens of the ophthalmic microscope to thereby cause the ophthalmic microscope to automatically adjust a focus level of the ophthalmic microscope on the ILM to accommodate for respiration-related movements of the patient during the ILM peeling maneuver.

12. The method of claim 11, wherein transmitting the focus control signal to the motor-driven lens of the ophthalmic microscope includes transmitting the focus control signal to an analog microscope.

13. The method of claim 11, wherein transmitting the focus control signal to the motor-driven lens of the ophthalmic microscope includes transmitting the focus control signal to a digital ophthalmic microscope.

14. The method of claim 11, wherein the low-coherence IR light source includes a low-coherence near-IR (NIR) light source, and wherein directing the low-coherence IR light toward the eye includes directing an NIR light beam toward the eye via the NIR light source.

15. The method of claim 11, wherein sensing the axial distance between the ophthalmic microscope and the ILM using the interferometer includes using an axial-scan ocular coherence tomography (A-scan OCT) device.

16. The method of claim 11, wherein sensing the axial distance between the ophthalmic microscope and the ILM using the interferometer includes using swept-source biometer.

17. The method of claim 11, further comprising:
generating the input signal in response to activation of a user interface.

18. The method of claim 17, wherein the user interface includes a manually-actuated foot pedal, and wherein generating the input signal in response to activation of the user interface includes detecting actuation of the manually-actuated foot pedal.

19. An electronic control unit (ECU) for use with an ophthalmic microscope having a motor-driven lens, the ECU comprising:
a processor; and
non-transitory, computer-readable storage medium on which instructions are recorded, wherein the instructions are executable by the processor to cause the ECU to:
command a low-coherence infrared (IR) light source of an interferometer to output a near-infrared (NIR) light beam toward an eye of a patient during an internal limiting membrane (ILM) peeling maneuver, the low-coherence IR light source including a super luminescent light-emitting diode (SLED);
command the interferometer to sense an axial distance between the ophthalmic microscope and an ILM of the eye, the interferometer being an axial-scan ocular coherence tomography (A-scan OCT) device or a swept-source biometer;
receive one or more distance signals from the interferometer, the one or more distance signals being indicative of the sensed axial distance; and
transmit a focus control signal to the motor-driven lens to thereby automatically adjust a focus level of the microscope on the ILM to accommodate for respiration-related movements of the patient during the ILM peeling maneuver.

20. The ECU of claim 19, wherein the instructions are executable by the processor to cause the ECU to:
receive an input signal from a user interface; and
in response to the input signal:
command the low-coherence IR light source to output the NIR light beam toward the eye;
command the interferometer to sense the axial distance;
receive the one or more distance signals from the interferometer, and
transmit the focus control signal to the motor-driven lens in response to the input signal.
